(19) United States
(12) Patent Application Publication
(10) Pub. No.: US 2008/0295574 A1
(43) Pub. Date: Dec. 4, 2008

(54) METHOD FOR FUEL ANALYSIS

(75) Inventors: Oliver Miersch-Wiemers, Schlosserstr. 2 (DE); Georg Mallebrein, Korntal-Muenchingen (DE); Stephan Uhl, Markgroeningen (DE)

Correspondence Address: MERCHANT & GOULD PC, P.O. BOX 2903, MINNEAPOLIS, MN, 55402-0903, US

(73) Assignee: Robert Bosch GmbH, Stuttgart (DE)

(21) Appl. No.: 12/105,839

(22) Filed: Apr. 18, 2008

(30) Foreign Application Priority Data
Apr. 27, 2007 (DE) 10 2007 019 992.0

(51) Int. Cl. G01N 33/487

(52) U.S. Cl. 73/23.31; 701/103

(57) ABSTRACT

The invention concerns a method for the fuel analysis in an internal combustion engine, which can be operated with different fuels containing ethanol and gasoline in varying compositions, wherein the gasoline content is ascertained in the fuel. In so doing, it is possible for especially the cold starting characteristics of the internal combustion engine to be improved, because the enrichment of the fuel mixture, which is necessary to achieve this improvement, can be optimally adjusted according to the gasoline content in the fuel.

### TECHNICAL FIELD

The invention concerns a method for fuel analysis in an internal combustion engine, which can be operated with different fuels containing ethanol and gasoline in varying compositions.

### BACKGROUND

Present-day internal combustion engines according to the principle of the gasoline engine are operated as a rule with a fuel extracted from crude oil and containing hydrocarbons such as regular or premium gasoline. Alternatively alcohol fuels extracted from plants, for example from sugar cane, such as ethanol are also increasingly being used.

A motor vehicle, which takes both kinds of fuel, is referred to as a vehicle capable of adapting to the fuel, or also as a “flexible fuel vehicle”, or in short as a “flex-fuel vehicle” (FFV) or as a flex-power vehicle. These types of vehicles can be operated with pure gasoline as well as with various similar fuels like, for example, ethanol, bioethanol or methanol-gas mixtures. Pure ethanol is denoted as E100-fuel. Pure gasoline is on the other hand denoted as E0-fuel. Any arbitrary mixture with xx % ethanol is denoted as Exx.

Because ethanol has a significantly smaller stoichiometric ratio during combustion when compared with gasoline (9.0 instead of 14.7), an increased injected fuel quantity is required in a stoichiometric engine operation with ethanol. This is made more complicated by the fact that arbitrary mixtures can occur in the fuel tank as a result of the fuels put in the tank. Information about a past fueling of the tank must at the latest be present when after filling the tank, new fuel with other characteristics has arrived at the fuel distributor rail, the so-called fuel rail, respectively at the fuel-delivery control system of the internal combustion engine. An exact knowledge of the ethanol content of the fuel significantly improves the drivability of the vehicle as well as the cold starting capability.

The following table provides an overview of the flex-fuels currently in use:

| | | | | Relative | |
|---|---|---|---|---|---|
| | Ethanol | Gasoline | Energy | Water | Distribution |
| Type | Content | Content | Content | Content | Region/Ctry. |
| E0 . . . 5 | max. 5% | min. 95% | 0% | 100% | Western |
| | anhydrous | | | | Europe |
| E10 | max. 10% | min. 90% | 0% | 97% | USA/Europe |
| | anhydrous | | | | being discussed |
| E24 | 24% | 76% | 0% | 91% | Brazil |
| | anhydrous | | | | (regular gasoline) |
| E85 | 85% | 15% | 0% | 70% | USA/Sweden |
| | anhydrous | | | | |
| E100 | 93% | 0% | 7% | 61% | Brazil (Alcohol) |

The engine management system of the flex-fuel vehicle must therefore adjust the engine mode of operation, especially the fuel injection mode of operation, respectively the fuel injection characteristic diagrams, to the corresponding fuel mixture ratio. Detection with certainty of the fuel mixture existing in the tank is required for this purpose. In so doing, it is assumed that the mixture ratio can only then change in the tank if a quantity of fuel has been added. For this reason a leading role is assigned to the fueling detection in a flex-fuel system.

State of the art is a detection, which detects the fuel level change in a vehicle at rest (fuel tank sender, signal at terminal 15). Fueling when the motor is running is not detected. The disadvantage thereby is that the fuel level measured in a vehicle at rest depends very greatly on the degree to which the vehicle is slanted. An improved fueling detection is described in a parallel application of the applicant (female).

It is disadvantageous according to the technical field that present-day flex-fuel systems especially determine only the ethanol content. Thus, a device to identify the alcohol-gas fuel mixtures in an internal combustion engine, which contains a sensor to measure the alcohol, is, for example, described in U.S. Pat. No. 5,179,926.

No additional information is, however, available about the remaining contents of the fuel. If, for example, the information were available that the fuel contained an ethanol content of 68%, this information would indeed be helpful with regard to the anti-knock properties of the fuel; however, it would not be sufficient with regard to the necessary enrichment of the fuel mixture during the cold starting phase of the internal combustion engine. For this purpose, it would be important to know if the remaining component of 32% consisted of summer or winter gasoline, which can have different characteristics, or possibly consisted of a mixture of gasoline and water.

It is, therefore, the task of the invention to provide a method, which allows for an accurate analysis of the fuel.

### SUMMARY

The task is thereby solved, in that the gasoline content is ascertained in the fuel. In so doing, it is possible to especially improve the cold starting characteristics of the internal combustion engine because the gasoline content predominantly determines the degree of required enrichment and thereby the ease in starting the engine. The required enrichment of the mixture can optimally be adjusted with accurate knowledge of the gasoline content, i.e. according to the gasoline content in the fuel. Advantages can also additionally be recorded for the transient operation of the internal combustion engine.

If, as provision is made in a preferred procedural variation, the ascertainment of the gasoline content is implemented in addition to a determination of the ethanol content, it is thereby possible for the proportional amount of a third component, which may be present, to be ascertained in the fuel. This, for example, can be the water content in the fuel. This can additionally be determined in an additional variation of the method. The ethanol content can in the process be ascertained by means of a sensor or from the signal flow of a lambda signal of an exhaust gas probe, as this is described in the parallel applications of the applicant (female).

Provision can be made in a preferred procedural variation for the gasoline content to be ascertained by means of a sensor, which results in particularly accurate measurements and additionally represents a redundancy during the fuel analysis.

Provision can also be made in another procedural variation for the gasoline content to be determined by means of a software algorithm. Additional sensors are not required, which is advantageous when costs are considered. A software solution of this kind can, for example, be implemented within the scope of an on-board diagnosis (OBD), wherein it can also be based on other already existing sensors.

If the gasoline content is determined from the vapor pressure of the fuel, the advantage follows, in that especially the vapor pressure of the fuel determines the cold starting capability of the internal combustion engine, and in that the vapor pressure of the fuel represents an important parameter in the mixture processing of ethanol-gasoline fuels.

The method with the variations described above can be employed in internal combustion engines, which can be operated with a fuel with a variable ethanol content, as is the case with the flex-fuel vehicle (FFV) and which especially have intake manifold fuel injection or direct gasoline injection. The method described can be implemented as a software solution and/or as a hardware solution and can at least be a part of the overriding engine management system.

1. A method for a fuel analysis in an internal combustion engine, which can be operated with different fuels containing ethanol and gasoline in varying compositions, the method comprising: ascertaining a gasoline content from a fuel.

2. A method according to claim 1, further comprising ascertaining an ethanol content.

3. A method according to claim 1, further comprising ascertaining a water content in the fuel.

4. A method according to claim 1, wherein ascertaining the gasoline content includes using a sensor.

5. A method according to claim 1, wherein determining the gasoline content includes using a software algorithm.

6. A method according to claim 1, wherein ascertaining the gasoline content includes ascertaining from a vapor pressure of the fuel.
